(19) United States
(12) Patent Application Publication
(10) Pub. No.: US 2021/0309752 A1
(43) Pub. Date: Oct. 7, 2021

(54) METHODS FOR TARGETING, INHIBITING, AND TREATING A TUMORAL MICROENVIRONMENT

(71) Applicant: THE TRUSTEES OF INDIANA UNIVERSITY, Bloomington, IN (US)

(72) Inventor: Sophie PACZESNY, Indianapolis, IN (US)

(21) Appl. No.: 17/261,998

(22) PCT Filed: Aug. 2, 2019
(86) PCT No.: PCT/US2019/044870

§ 371 Date: Jan. 21, 2021

Related U.S. Application Data
(60) Provisional application No. 62/714,298, filed on Aug. 3, 2018.

(51) Int. Cl. C07K 16/28

(52) U.S. Cl. CPC C07K 16/2866; A61K 2039/505

(57) ABSTRACT

The present application relates to methods for targeting and/or inhibiting a tumoral microenvironment and/or tumor cells. More specifically, the present disclosure is directed to a method of targeting a tumor microenvironment of a subject, the method comprises, consists of, or consists essentially of inhibiting ST2* regulatory T cells present in the tumor microenvironment. The methods of the present disclosure further comprise blocking the IL-33/ST2 pathway.

### CROSS-REFERENCE TO RELATED APPLICATIONS

This application claims priority to and the benefit of U.S. Provisional Patent Application No. 62/714,298, filed Aug. 3, 2018, the disclosure of which is hereby expressly incorporated herein by reference in its entirety.

### FIELD OF THE PRESENT APPLICATION

The present application relates to methods for targeting, inhibiting, and/or treating a tumoral microenvironment and tumor cells comprising blocking the IL-33/ST2 pathway.

### BACKGROUND

Acute myeloid leukemia (AML), which in 2015 affected >20,000 patients and led to >10,000 deaths in the US alone (1-3), constitutes a critical unmet therapeutic need. Among all childhood cancers, acute myeloid leukemia (AML) continues to have the lowest 5-year survival, at <75%. Other childhood cancers that have not reached 5-year survival, in contrast to acute lymphoblastic leukemia or lymphoma with >95% response rate, are brain tumors, osteosarcomas, soft tissue sarcomas and neuroblastoma although there are substantial better therapeutic options for the later, particularly with anti-GD 2 antibody immunotherapy even eliminating the need for auto-transplant. Therefore, there is an unmet need for novel immunotherapies for these childhood cancers with lower survival rates.

The standard of treatment of AML has remained relatively unchanged for >20 years. Although chimeric antigen receptor (CAR) T cells targeted against CD33 show promise in acute and chronic lymphoid leukemia, their use in AML requires subsequent hematopoietic cell transplantation (HCT) due to myelotoxicity to normal myeloid cells. While these and other promising approaches have emerged, such as bispecific antibodies directed toward CD33 or CD123 that do not require transplantation or HCT, there is still a need for new and more specific targets of both the tumor and tumor microenvironment.

To date, there are two mainstay approaches to mitigating pediatric cancers: (i) targeting tumor antigen, such as CD19 CAR T cells in acute lymphoblastic leukemia or anti-GD2 in neuroblastoma, and (ii) targeting the inhibitory immune microenvironment to render it more cytotoxic against the tumor with checkpoint inhibitors such as anti-CTLA4, anti-PD1 and anti-PD-L1, and melanoma and lung cancers respond well to this approach as these tumors bear a high tumor antigen burden. However, the later approach has, for the most, not been useful in pediatrics cancers due to their low tumoral hypermutations rate, with the exception of the recent described germline biallelic mismatch repair where checkpoint inhibitors are highly efficient.

CD4+ regulatory T cells (Tregs) play a critical role in the induction, orchestration, and maintenance of the tolerance of immune responses within the host. They largely accomplish this role by secreting or interacting with cytokines and other molecules that sculpt the inflammatory environment and by suppressing other immune cells in the site of inflammation, or by directly interacting with immune cells to influence their differentiation. The ability of Tregs to acquire unique programs of differentiation is based on their responsiveness to the inflammatory environment, which alters transcriptional activity to direct cell fate. The classic Tregs are thymus-derived and express the transcription factor forkhead box P3 (FOXP3), which is a key intracellular marker and a crucial functional factor for Tregs.

Tregs are required to maintain immune homeostasis and prevent excessive tissue damage, however they can be deleterious in cancer through suppression of anti-tumor immunity. High numbers of Tregs and a low CD8+ T cell:Treg ratio are considered poor prognostic factors for many tumor types, including melanoma, head and neck squamous cell carcinoma, ovarian cancer, and colorectal carcinoma. This phenomenon has been well established in human and experimental solid tumors; however, this has not been shown in the malignant bone marrow (BM) niche and other leukemia target organs. Recent research has demonstrated that Treg-mediated immunosuppression is one of the crucial tumor immune-evasion mechanisms and the main obstacle of successful tumor immunotherapy.

Although targeting intratumoral Tregs could be an effective therapeutic approach for multiple tumor types, perturbation of peripheral Treg number or function could lead to life-threatening autoimmune or inflammatory complications. Therefore, identifying pathways that could be targeted to selectively undermine intratumoral Tregs is essential. Indeed, current therapeutic approaches to delete Tregs (e.g., anti-CTLA4, anti-CD27, etc.) use general markers on both peripheral and intratumoral Tregs.

Tregs sense the tumor microenvironment by responding to multiple cytokine signals in the inflammatory milieu. One such cytokine of importance, particularly in the tumor microenvironment, is the interleukin (IL)-33, a member of the IL-1 family of cytokines that is released upon tissue stress or damage to operate as an alarmin and has recently been shown to induce tumor pathogenesis in myeloproliferative disorders, a preleukemic state, and in colon cancer. However, the role of IL-33 and STimulation-2 (ST2), its only known receptor, was not evaluated. Specifically, the mechanisms underlying how the ST2/IL-33 pathway works to govern Treg differentiation versus type 1 T helper and CD8+ cytotoxic T cell antitumoral effects is not known. Furthermore, ST2⁺ Tregs have been shown to be tissue-restricted in the normal and inflammatory microenvironment and are not found in the periphery. The presence of ST2⁺ Tregs and its likely enrichment in the microenvironment of solid or liquid tumors has not been explored at all so far.

It has been shown that stromal cell-derived IL-33 stimulated the secretion of cytokines and growth factors by myeloid and non-hematopoietic cells of the bone marrow (BM), resulting in myeloproliferation in SHIP-deficient animal. Additionally, in the transgenic JAK2V617F model, the onset of myeloproliferative neoplasms was delayed in animals lacking IL-33, and increased numbers of IL-33-expressing cells were detected in human BM of patients with myeloproliferative diseases. In a more recent study, using immunohistochemistry on 713 resected human colorectal cancer (CRC) specimens, the group above showed that both IL-33 and its receptor ST2 were expressed in early-stage human CRCs and thus, induced CRC and other myeloproliferative diseases. In a mouse model of CRC, ST2-deficiency protected from tumor development and activation of IL-33/ST2 signaling compromised the integrity of the intestinal barrier and triggered the production of pro-tumorigenic IL-6 by immune cells. Together, this data revealed a tumor-promoting role of IL-33/ST2 signaling in CRC.

Applicant has also generated previous data in a model of mice with MLL-AF9 AML that received allogeneic HCT (allo-HCT) as a potentially curative option through the graft-versus-leukemia (GVL) activity with concomitant anti-ST2 treatment for graft-versus-host disease protection, 80% of animals survived as compared to 0% and 20% of tumor-bearing syngeneic animals and nontreated tumor-bearing allo-HCT recipient animals, respectively. This survival benefit was higher than expected with the GVHD protection alone, suggesting a potential direct targeting of leukemic cells with anti-ST2 treatment.

### SUMMARY OF INVENTION

The present application relates to methods for targeting and/or inhibiting a tumoral microenvironment and/or tumor cells. More specifically, the present disclosure is directed to a method of targeting a tumor microenvironment of a subject, the method comprises, consists of, or consists essentially of inhibiting ST2⁺ regulatory T cells present in the tumor microenvironment. The inhibition of ST2⁺ regulatory T cells of the claimed method may comprise destroying ST2⁺ tumor cells directly.

The method may also comprise administering one or more antibodies to the tumor microenvironment of the subject or by other means. For example, the method may comprise use of bispecific antibodies targeting CD4/ST2, as well as CART cells, NK cells, and/or induced pluripotent cells targeting ST2, or combinations thereof.

The method may also comprise a means for blocking the IL-33/ST2 pathway in the tumor microenvironment or in the tumor tissue. The means for blocking the IL-33/ST2 pathway may comprise: a) administering one or more neutralizing antibodies to the subject, b) bispecific antibodies targeting CD4/ST2, and c) CART cells, NK cells, or induced pluripotent cells targeting ST2, or combination thereof. The claimed method also increases tumor immunity in the malignant bone marrow niche or solid tumor/tissue microenvironment.

The tumor microenvironment comprises, consists essentially of, or consists of any and all cells associated with the tumor but that are not tumor cells. The tumor microenvironment of the present method comprises, consists essentially of, or consists of cells of tumors selected from the group consisting of liquid tumors or solid tumors. The tumor microenvironment may comprise a malignant bone marrow niche, such as for liquid tumors. The tumor microenvironment may also comprise tissues surrounding tumors, including for solid tumors, such as sarcomas.

The tumor microenvironment may also comprise tumors of cancer disease. The cancer disease of the present method may be selected from the group consisting of leukemia, lymphoma, osteosarcomas, neuroblastoma, colorectal cancer, and soft tissue sarcomas. The leukemia of the present method may be acute myeloid leukemia (AML). Finally, the subject of the claimed method may be an adult patient or a pediatric patient.

The present application also relates to methods for treating cancer in a subject. More specifically, the present disclosure is directed to a method of treating cancer in a subject, the method comprising: a) administering one or more antibodies to the tumor microenvironment of the subject and b) inhibiting ST2⁺ regulatory T cells present in the tumor microenvironment. The method may further comprise destroying ST2⁺ tumor cells located in the tumor microenvironment. The method may also comprise blocking the IL-33/ST2 pathway in the tumor microenvironment, wherein the tumor microenvironment comprises a malignant bone marrow niche for liquid tumors or the tissues surrounding tumors for solid tumors such as sarcomas. In addition, the claimed method increases tumor immunity in the malignant bone marrow niche or solid tumors microenvironment.

The tumor microenvironment of the present method may comprise tumors selected from the group consisting of liquid tumors (e.g., cancer of bone marrow) or solid tumors (e.g., found in any cancerous tissue). The tumor microenvironment may also comprise tumors of cancer disease. The cancer disease of the present method may be selected from the group consisting of leukemia, lymphoma, osteosarcomas, neuroblastoma, colorectal cancer, and soft tissue sarcomas. The leukemia of the present method may be acute myeloid leukemia (AML). Finally, the subject of the claimed method may be an adult patient or a pediatric patient

### BRIEF DESCRIPTION OF THE DRAWINGS

FIG. 1A is a graph and FIG. 1D is a pie chart showing that administration of anti-ST2 mAb preserved GVL activity and resulted in significantly improved leukemia-free survival in contrast to mice that received syngeneic HCT and died (FIG. 1B) of leukemia or mice that were treated with the isotype control (i.e., IgG) and died of GVHD (FIG. 1C).

FIG. 2 is a schematic of the model of cytokine balance in the development of ST2⁺Tregs and ST2⁺ tumoral cells in the malignant bone marrow (BM)-niche with or without ST2/IL-33 blockade.

FIG. 3A is a graph showing sorted Foxp3GFP+ Tbet^{-/-} Tregs vs. WT Tregs from bone marrow (BM) cells. Nanostring analysis was performed with the nCounter Analysis System at NanoString Technologies. The nCounter Mouse Immunology Kit, which includes 561 immunology-related mouse genes was used. FIG. 3B shows representative plots and Mean±SEM graphs of frequencies of ST2⁺ Tregs analyzed by flow cytometry in the BM of naïve B6 WT or Tbet^{-/-} mice gated on CD4 T cells, n=3. FIG. 3C is a graph showing mean±SEM graphs of frequencies of Tregs analyzed by flow cytometry in different organs (spleen, liver, BM, and peripheral blood) of naïve B6 WT or Tbet^{-/-} mice gated on CD4 T cells, n=3.

FIG. 4 shows representative FACS plots and mean±SEM graphs of Tregs cultured in anti-CD3 mAb coated 96 well plates at 0.1M Tregs per well in media with different cytokines conditions: IL-2 (2 ng/mL) or IL-2 (2 ng/mL)+ IL-33 (20 ng/mL) or IL2 (2 ng/mL)+ IL-33 (20 ng/mL)+ IFN-γ (40 ng/mL). After 3 days culture, cells are collected for FACS (ST2, FoxP3 staining), n=3.

FIG. 5A is a schematic showing AML mouse models used employed to transplant T cells from ST2^{-/- Foxp3GFP} or WT syngeneic animal into mice with tumors and FIG. 5B is a schematic showing AML mouse models used employed to transplant T cells or AML cells from ST2^{-/- Foxp3GFP} or WT syngeneic animal into B6 host mice.

FIG. 6A shows representative FACS plots of Foxp3egfp and KLRG1 expressions gated on live CD3+ cells, and mean±SEM bar graphs showing the frequencies of Foxp3+KLRG1+ cells from B6 mice receiving 105 or 106 syngeneic MLL-AF9 GFP+ AML cells with 2×106 B6 Foxp3 GFP+ WT syngeneic T cells (n=3). Representative FACS plots and mean±SEM bar graphs showing the frequencies of PD-1+IFNγ+ cells from the same B6 mice as above (n=3) and FIG. 6B shows Representative FACS plots and mean±SEM bar graphs showing ST2+Foxp3+ T cells and KLRG1 or CD69 activation markers on Tregs and IFNgamma on Foxp3− T cells in syngeneic HCT and leukemia bearing C1498Tdtom mice receiving 106 syngeneic C1498Tdtom AML cells with WT or ST2−/− syngeneic T cells.

FIG. 7A shows representative FACS plots and mean±SEM bar graphs showing MLL-AF9 GFP+ AML cells proliferation in B6 mice receiving 105 or 106 syngeneic MLL-AF9 GFP+ AML cells with 2×106 B6 Foxp3 GFP+ WT or ST2−/− syngeneic T cells (gated on live CD3− cells, n=3) and FIG. 7B shows representative FACS plots and mean±SEM bar graphs showing syngeneic HCT and leukemia bearing C1498Tdtom mice receiving 106 syngeneic C1498Tdtom AML cells with WT or ST2−/− syngeneic T cells.

FIG. 8A shows representative FACS plots and mean±SEM bar graphs showing the frequencies of Foxp3+KLRG1+ cells and PD-1+IFNγ+ cells in B6 mice receiving 105 or 106 syngeneic MLL-AF9 GFP+ AML cells with 2×106 B6 Foxp3 GFP+ WT or ST2−/− syngeneic T cells (gated on live CD3+ cells, n=3) and FIG. 8B shows representative FACS plots and mean±SEM bar graphs showing the activation marker CD69 on CD8+ T cells.

FIG. 9 shows representative FACS plots showing the frequencies of ST2+ cells and IL-33+ cells in various murine and human AML cell lines.

FIG. 10 shows representative FACS plots showing ST2+, IL-33+, and PDL1 expression in classical stem cells defined as CD34+ (positive) cells and CD38− (negative).

FIG. 11 shows representative FACS plots showing ST2+, IL-33+, and PDL1 expression in primitive or classical stem cells that are CD34+ (positive), CD38− (negative), CD90+, and CD45RA+ are “true” leukemic stem cells.

FIG. 12 shows representative FACS plots of Foxp3egfp and KLRG1 expressions gated on live CD4⁺ and CD8 cells⁺, and mean±SEM bar graphs showing the frequencies of KLRG1⁺ ST2⁺ Foxp3⁺ Tregs from three patients with refractory AML as compared to three patients with first complete remission (CR1). Representative FACS plots showing the frequencies of PD-1⁺IFNγ+ cells from the same patients as above (n=3 per group).

FIG. 13A is a graph showing an antitumor role of ST2 in a murine CRC xenograft model. FIG. 13B are photographs that demonstrate ST2^{-/-} mice display a significant decrease in CRC tumor growth (n=10).

FIG. 14A shows representative FACS plots and mean±SEM bar graphs showing the frequencies of ST2+ and IL-33+ cells among CD45+CD33+ primary leukemic cells. FIG. 14B shows graphs showing the frequencies of ST2+ and IL-33+ cells in three patients with refractory AML comparing to three patients in CR1 (n=3 per group).

FIG. 15 is a graph showing survival curves (Kaplan Meier) by high and low ST2 expression on AML tumoral cells.

FIG. 16 shows representative tSNE plots from a healthy donor (among 10 HD tested) for ST2 expression and CD4 and B cells markers CD19 and CD20.

### DETAILED DESCRIPTION

The present application relates to methods for targeting and/or inhibiting a tumoral microenvironment and/or tumor cells. The methods of the present disclosure comprise, consist of, and/or consist essentially of blocking the IL-33/ST2 pathway. ST2 is expressed by tumor infiltrating Tregs in samples from patients with cancer, including childhood cancers, and is critical for their inhibitory function in the tumor microenvironment. More specifically, the methods of the present disclosure are based on the understanding that: (ii) blocking the IL-33/ST2 pathway with neutralizing antibodies or other means (see above) in the microenvironment of tumors, including AML and solid tumors, will reduce ST2⁺ Tregs infiltration, increase the CD8+ T cell:Treg ratio, and release the brake on the anti-tumor response.

Based on this knowledge, the present methods comprise, consist of, and/or consist essentially of neutralizing antibodies directed at ST2 in tumors, such as AML and solid tumors that may be directly tumoricidal. Thus, the methods of the present disclosure target the IL-33/ST2 pathway can be therapeutic through a mechanism, such as a dual mechanism comprising, consisting of, and/or consisting essentially of 1) targeting and/or blocking of tumor infiltrating ST2⁺ Tregs in the tumor microenvironment as opposed to targeting a global marker of Tregs and 2) destruction of ST2⁺ tumor cells by exploiting ST2-specific antibodies.

More specifically, the methods of the present disclosure comprise, consist of, or consist essentially of 1) blocking ST2+ Tregs that increase tumor immunity in the malignant BM niche (e.g., AML), which may be used as a novel antitumoral immunotherapy and 2) combination of transcriptome, proteome, and systems biology analyses looking at activity of specific gene markers (e.g., FOXP3, ST2, and/or IL-33) and their relation to Tbet and/or interferon-y levels in patients' BM aspirates as prognostic biomarkers for clinical outcomes of disease, such as adult and/or pediatric cancers. For example, the present methods and composition may be used to ameliorate treatment of AML via modulation of the immune system. The clinical, therapeutic, and/or diagnostic mechanism employed by the present methods is applicable to the tumor micro-environment in other hematological malignant diseases, as well as solid tumors.

The methods of the present disclosure also provide greater clarity on how ST2/IL-33 signaling potentiates the activity of Tregs and how elements of type 1 immunity counteract those effects which have so far been underexplored. Another important feature of the present methods is the modulation of ST2 and its influence on the intratumoral Tregs compartment and growth of the tumor itself. The data provided herein support pursuing ST2⁺ Tregs as targets for immunotherapeutic intervention. In addition, the methods of the present disclosure incorporate ST2 as a viable tumor target and have huge implications, particularly when the relative expression among tumors versus normal tissues can be precisely mapped. ST2 targeting may also be combined with other immune checkpoint inhibitors or T-cell based therapies, including CART cells and T-cell engaging bispecific antibodies.

Targeting ST2 as an immune checkpoint in cancer is novel. Exploiting ST2 as a tumor target is also new. When the same marker ST2 is found on both Tregs in the tumor microenvironment and on tumor cells, dual targeting of the seed and soil may occur, which is a unique approach.

Furthermore, while the biology to understand ST2⁺ Tregs in colitis and diabetes-induced inflammation is emerging, the function of ST2⁺ Tregs in the tumor environment comprising the tumoral niche, particularly the malignant tumoral niche, is virtually unexplored. Another unexpected result of the present methods lie in their ability to determine and demonstrate the role of ST2⁺ Tregs in both the malignant niche using several complementary approaches, along with a comprehensive examination of the extent of tumoral progression in the presence or absence of ST2⁺ Tregs. Similarly, mechanisms leading to the overexpression of the ST2/IL-33 pathway on tumoral cells are generally unknown with the exception of colorectal cancer (CRC). However, the present methods provide a necessary advance.

Finally, for translation purposes, the methods of the present disclosure will identify and/or generate novel anti-ST2 neutralizing antibodies as reagents and as pharmaceutical candidates. The present methods will greatly benefit all cancer patients with potential applications for other adult cancers and pediatric cancer diagnoses worldwide. Moreover, the present methods also comprise, consist of, and/or consist essentially of the targeting, analysis, and/or inhibition of both liquid and solid tumors, with methods and on equipment used in both immunology and antibody engineering, while exploiting new and established preclinical mouse models to accelerate clinical translation of anti-ST2 antibody candidates.

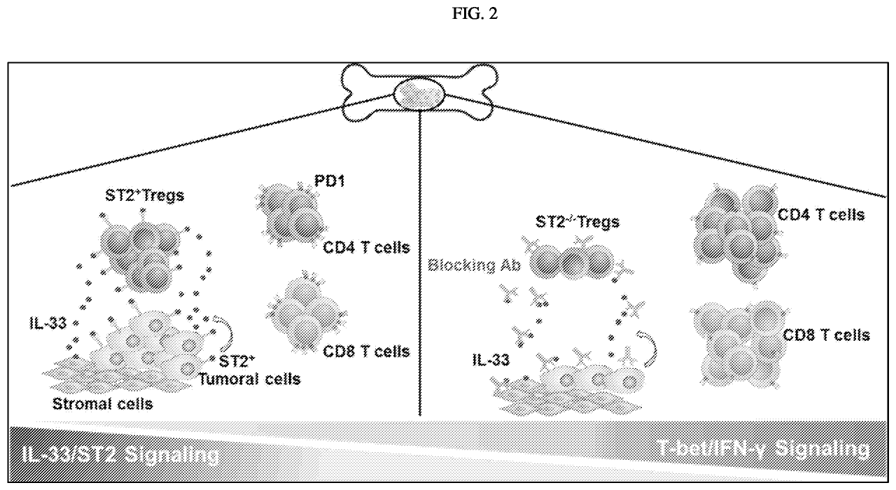

The present methods are developed based on the understanding that the balance between ST2/IL-33 and type 1 signaling influences Treg proliferation/function in the nonmalignant and/or malignant niche and that blockade of ST2⁺ Tregs will decrease tumor proliferation, particularly in the malignant BM niche. For example, FIG. 2 is a schematic of the model of cytokine balance in the development of ST2⁺ Tregs in the malignant bone marrow (BM) niche, in the case of cancers, such as leukemia, with or without anti-ST2 treatment (see FIG. 2).

T-bet^{-/-} mice exhibit increased numbers of ST2⁺ Tregs in the BM niche compared to WT mice. The potency of ST2⁺ Tregs in models of inflammatory diseases has been reported by the applicant and several other groups. However, a key feature of the present methods is the identification of components of T cell-driven inflammation that limit the expansion of ST2+ Tregs in the BM niche. For example, IFN-γ-producing CD4+ helper T cells (Th1) and CD8+ cytotoxic (Tc1) express the transcription factor T-bet, which enforces stable IFN-γ production.

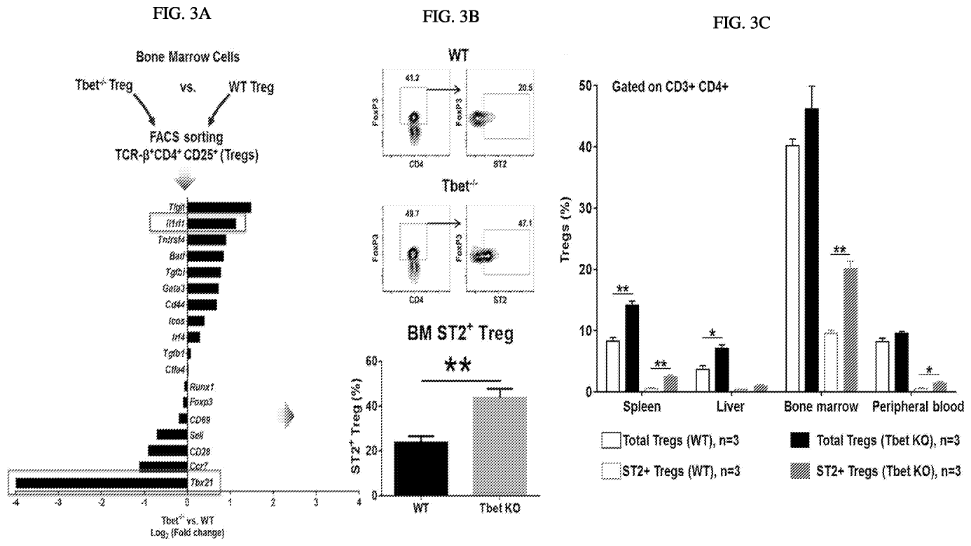

Thus, the present disclosure is directed to methods, wherein type 1 immunity will inhibit ST2⁺ Treg expansion, particularly within the BM niche. Initially, the genetic signature of BM cells from mice that are genetically deficient in T-bet versus naïve B6 WT, using analysis (e.g., a NanoString) of >500 immune-related genes was performed. The transcriptome of Treg-related genes were further analyzed and subsets of genes that were specifically induced in T-bet^{-/-} cells were identified (FIG. 3A). Il1rl1, the gene for ST2, was one of the most differentially expressed genes. Additional genes associated with Treg function, including Tigit, BATF, GATA-3, IFN regulatory factor 4 (Irf4), and CTLA-4 were observed to be enriched within the BM cells from T-bet^{-/-} mice (FIG. 3A). In agreement with that observation, mice that are genetically deficient in T-bet have elevated ST2⁺ Tregs at steady state in the BM (FIG. 3B). Importantly, at steady state only 1% of all hematopoietic cells are CD3⁺CD4⁺ in the BM, while 40% are Tregs. 25% of the Tregs are ST2⁺ Tregs. This final percentage of ST2⁺ Tregs is 10-fold higher than the percentages of Tregs that are ST2⁺ in the spleen, liver, and peripheral blood of mice. This percentage is also significantly increased in T-bet^{-/-} mice (FIG. 3C).

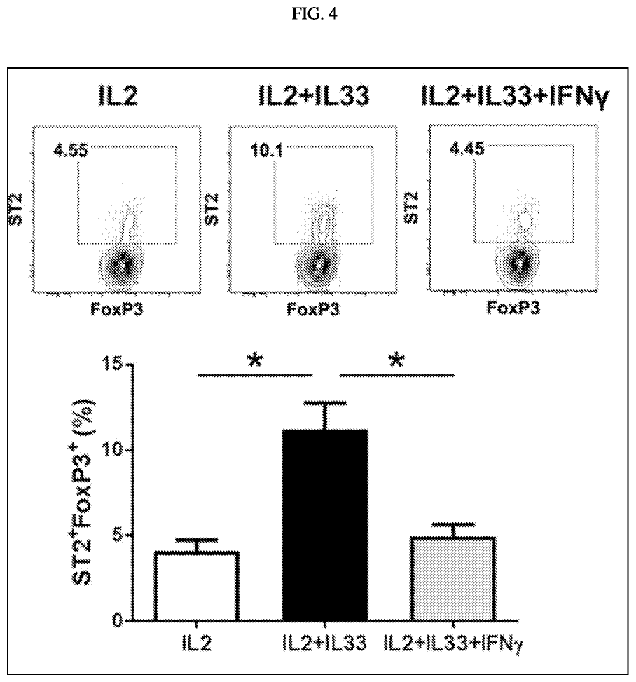

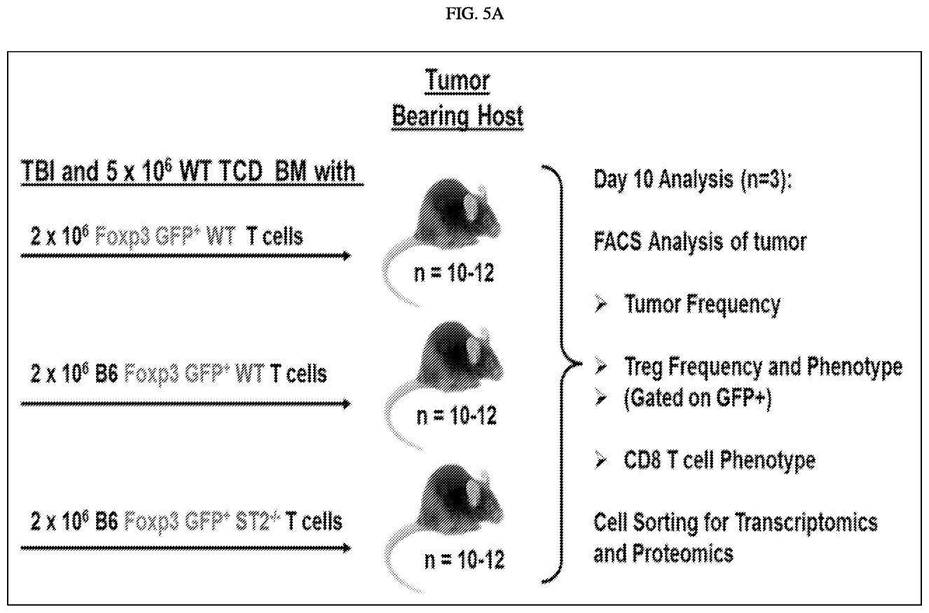

IFN-γ inhibits the ST2⁺ Treg expansion induced by IL-33. In vitro experiments were performed to determine if addition of IFN-γ limits the expansion of ST2⁺ Tregs induced by IL-33. Tregs were purified from mice (e.g., B6 mice) with a Miltenyi kit and expanded with IL-2 and IL-33 as previously described. The results confirmed that IL-33 is crucial for ST2⁺ Treg expansion. This expansion was inhibited by the addition of IFN-γ to the culture medium (FIG. 4).

Since IL-33 signaling enhanced the development of ST2⁺ Tregs in vitro, the absence of T-bet/IFN-γ signaling enhanced the development of ST2⁺ Tregs in the BM niche, and IL-33 can be secreted in the malignant BM niche, ST2⁺ Tregs in vivo in the malignant BM niche was also examined with the understanding that increased percentages of ST2⁺ Tregs with decreased type 1 immunity would be present in the malignant BM niche. Several mice models were investigated, including mice models of AML in the syngeneic HCT or leukemia-bearing mouse settings (e.g., cells).

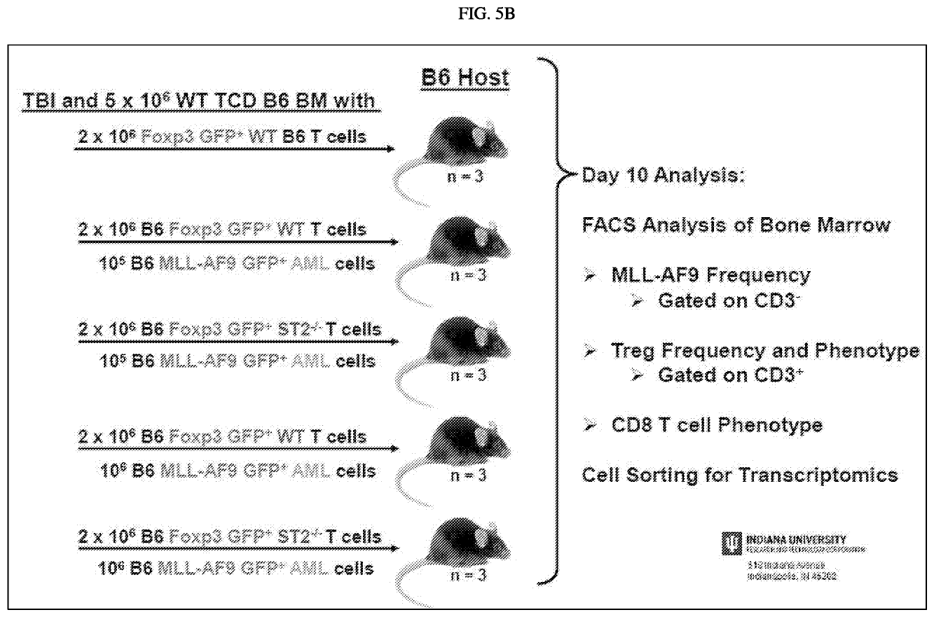

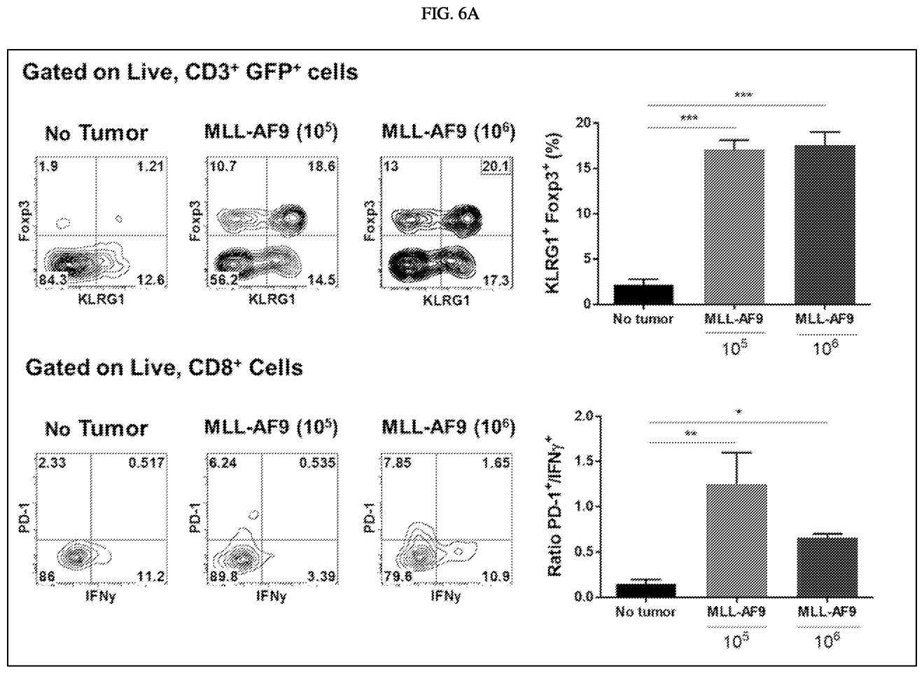

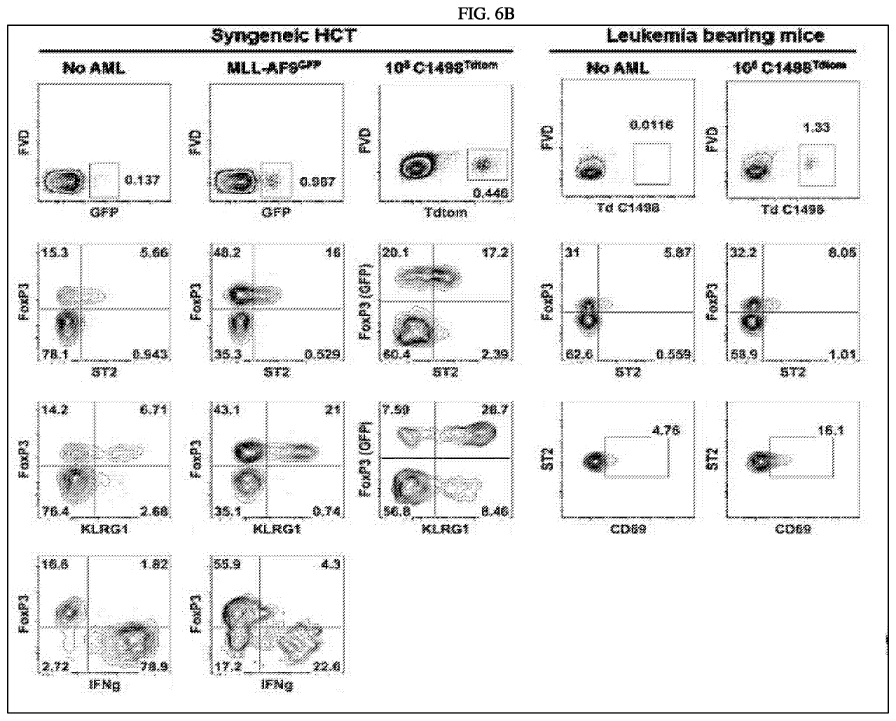

For example, T cells, BM cells, and 10⁴ MLL-AF9GFP AML cells on the C3H.SW background were transplanted into lethally irradiated C3H.SW mice. Alternatively, the present methods comprise, consist of, or consist essentially of analysis of syngeneic HCT mouse model with multiple doses (e.g., two, two or more, three, three or more, etc.) of 10⁵ and 10⁶ MLL-AF9^{GFP} AML cells on the B6 background that were transplanted into lethally irradiated B6 mice. T-cell subsets and leukemic cells were examined in the BM niche at day 10 post-transplantation for these two models (FIG. 5B). It was observed that 30% and 33% of leukemia-infiltrating CD3⁺ T cells were Foxp3^{+ GFP} Tregs, while the nonmalignant niche had only 3% Tregs. Furthermore, ˜20% of the Tregs observed expressed the activation markers KLRG1 versus 1% in normal BM (FIG. 6A, top panel). It was also demonstrated that IFN-γ producing CD8+ T cells were decreased in the malignant niche and express more PD-1, an exhaustion marker as compared to the nonmalignant BM niche (FIG. 6A, bottom panel).

In addition, 10⁵C1498^{Tdtom} AML cells were injected into naïve B6 mice to generate leukemia-bearing mice and leukemic cells and T-cell subsets were examined in the BM niche at day 15 post-injection (FIG. 5A). It was found that 16% and 17% of leukemia-infiltrating CD4+ T cells were Foxp3+ cells expressing ST2 at day 10 post-HCT when less than 1% of leukemia cells were infiltrating the BM niche, while the nonmalignant niche had only 5% ST2+ Tregs. Furthermore, ˜25% of these Tregs expressed the activation markers KLRG1 and CD69 versus 6% in normal BM (FIG. 6B). In the BM niche of leukemia-bearing animals, up to 11% ST2+ Tregs were identified as compared to 5% in WT BM, with a 4-fold increase in Tregs expressing activation markers as compared to the nonmalignant BM niche (FIG. 6B). This suggests that the pathways that induce ST2+ Tregs in the steady state BM niche have similar effects in the malignant BM niche.

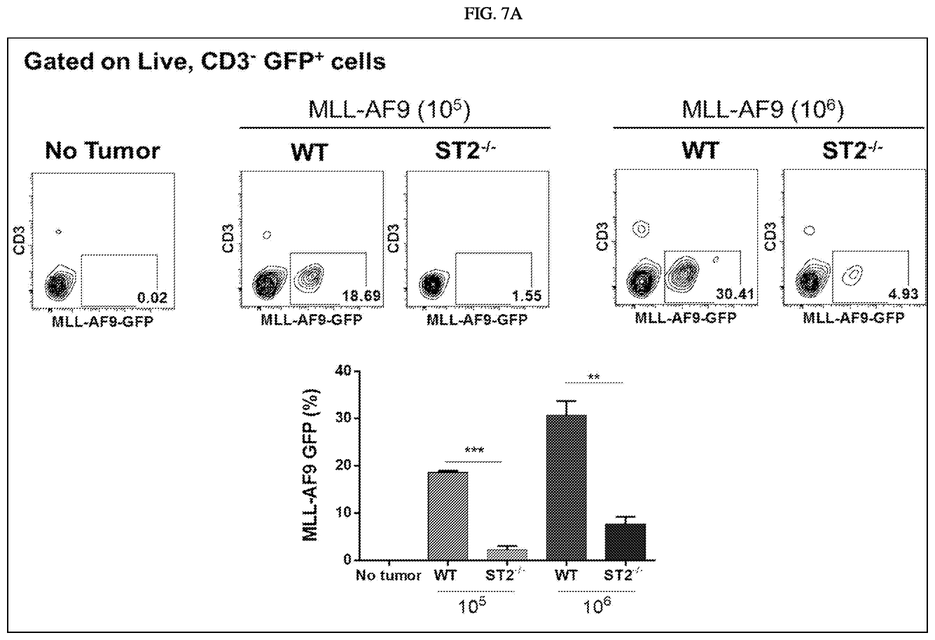

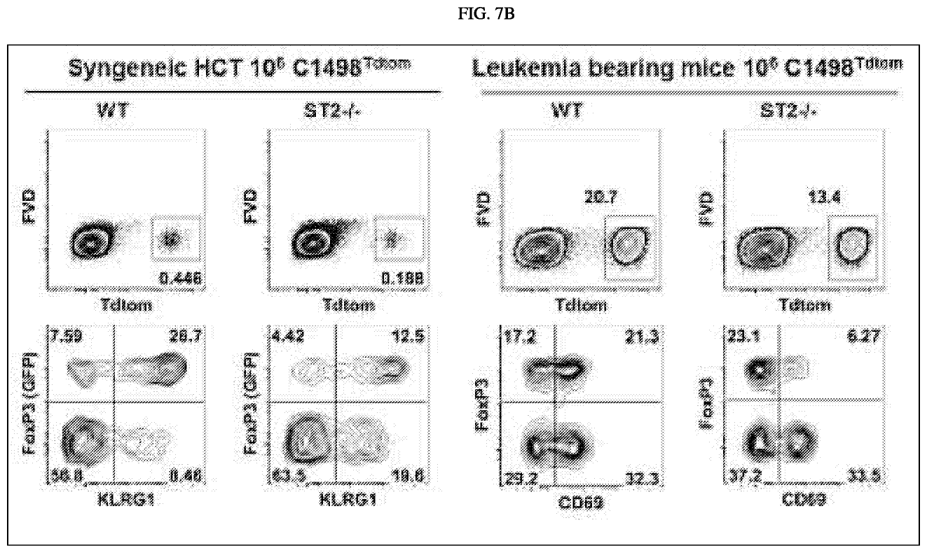

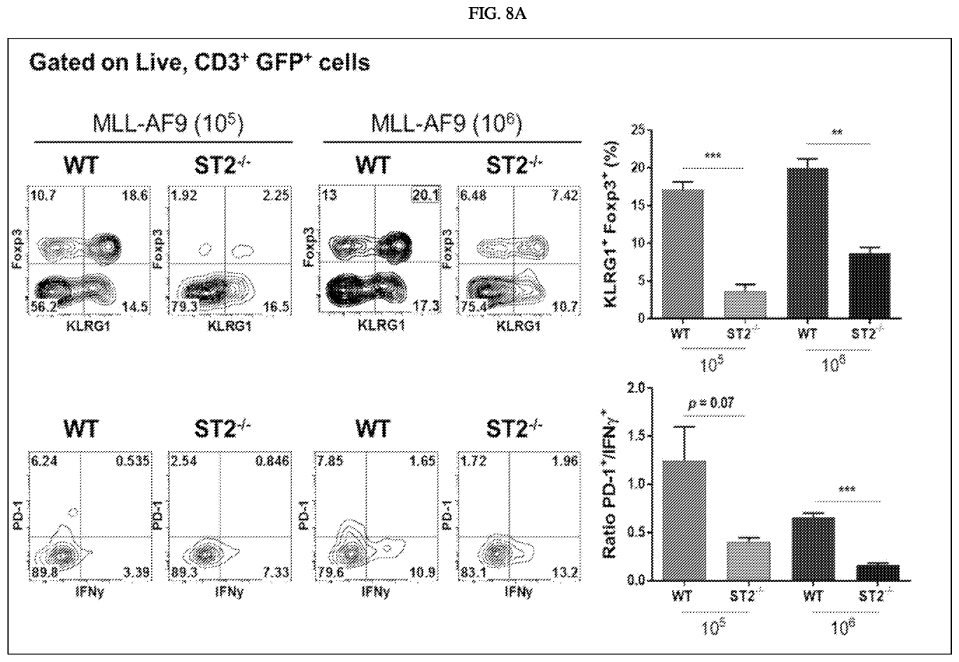

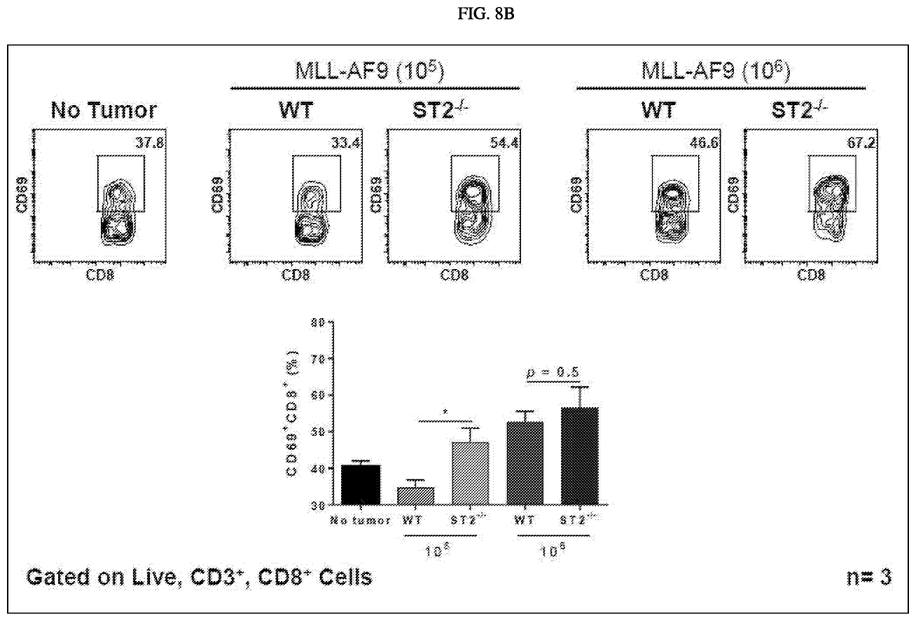

It was also observed that genetic blockade of ST2⁺ Tregs decreased tumor proliferation in the malignant BM niche, particularly in the syngeneic HCT cells and ST2^{-/- Foxp3GFP} cells on B6 background (FIG. 7A), as well as the C1498^{Tdttom} AML models used for this evaluation (FIG. 7B). When T cells from ST2^{-/- Foxp3GFP} were transplanted into mice with leukemia (e.g., the syngeneic model), the AML cells proliferated less in the BM niche of ST2^{-/-} animals receiving ST2^{-/- Foxp3GFP} cells than when receiving wildtype (WT) T cells (FIG. 7B, top panel). Mice receiving WT T cells had more activated as measured by the expression of KLRG1 and/or CD69 Tregs than mice receiving ST2^{-/- Foxp3GFP} (FIG. 7B, bottom panel). Similarly in the syngeneic model with 10⁴ or 10⁵MLL-AF9GFP AML cells (FIG. 5B), the total Treg population was decreased and less activated, as measured by the expression of KLRG1, in the BM niche of both models of ST2^{-/- Foxp3GFP} mice, while CD8⁺ T cells were producing more IFN-γ and expressed less PD-1 (FIG. 8A), and CD8⁺ T cells were more activated, as measured by the expression of CD69 (FIG. 8B).

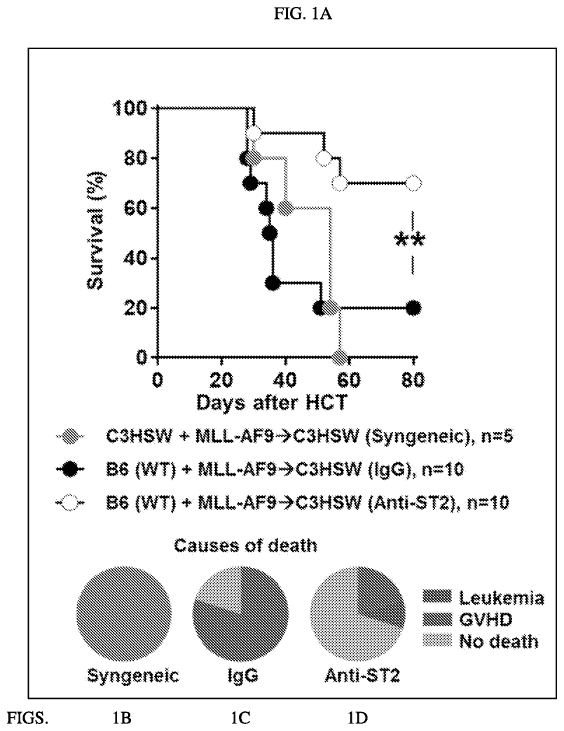

ST2 blockade increases antitumoral cytotoxicity following allogeneic HCT possibly through a direct ST2 targeting of ST2 on leukemic cells. It was also observed that the present methods may comprise, consist of, or consist essentially of administration of anti-ST2 mAb that preserved substantial GVL activity and resulted in significantly improved leukemia-free survival in contrast to mice that received syngeneic HCT and died of leukemia or mice that were treated with the isotype control and died of GVHD (FIG. 1). This survival benefit was higher than expected with the sole GVHD protection (average of 50% at day 80 in this model) suggesting a potential increase in the antitumoral activity with the anti-ST2 treatment through a direct ST2 targeting of ST2 on leukemic cells that will implement in the present methods.

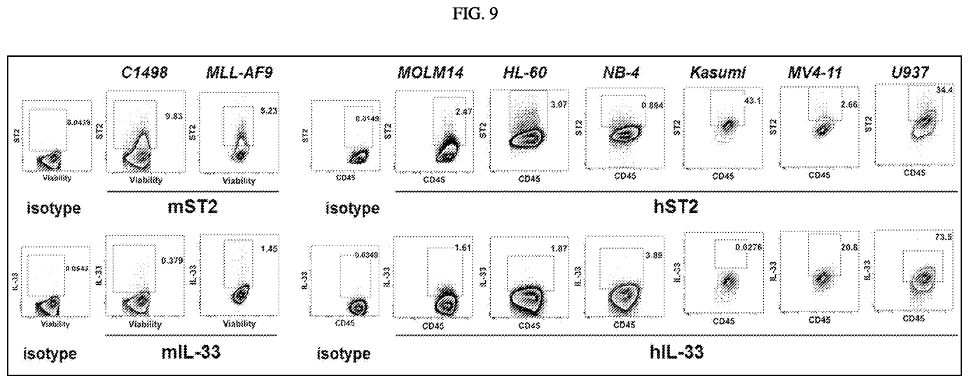

The expression of ST2 on the tumoral surface and intracellular IL-33 of various murine and human cell lines, including cancer cell lines, such as AML cells, was investigated. All cell lines tested expressed a degree of ST2 and/or IL-33 (FIG. 9). On average 3-10% of cells expressed ST2, but with some cell lines expressing up to 43% (e.g., Kasumi and U937). On average 1 to 3% of cells expressed IL-33, but again with some cell lines expressing up to 73% (U937 and MV4-11). ST2 and IL-33 expressions are not always correlated suggesting that some tumoral cells may be activated through a positive auto-feedback loop but not always. As for CRC, it is possible that ST2/IL-33 is an early event which make it even more attractive as a therapeutic target.

Overall, the preliminary data demonstrate that ST2⁺ Tregs differentiate in the presence of unique cytokine combinations and that these cells are present in vivo in the BM niche and increased during AML infiltration of the BM. Neutralizing ST2⁺ Tregs decreased the leukemia infiltration of the BM, further confirming their pivotal role in the malignant BM niche. Further, CRC tumoral cells and AML cells express ST2. Despite these advances, it is still unclear how IL-33/ST2 signaling confers potent Tregs function/expansion and overexpression on tumoral cells in the malignant niche. The experiments shown here were designed to explore these questions and provide new insights into the function of ST2⁺ Tregs in the BM niche and the potential usefulness of their blockade to treat leukemia and solid tumors.

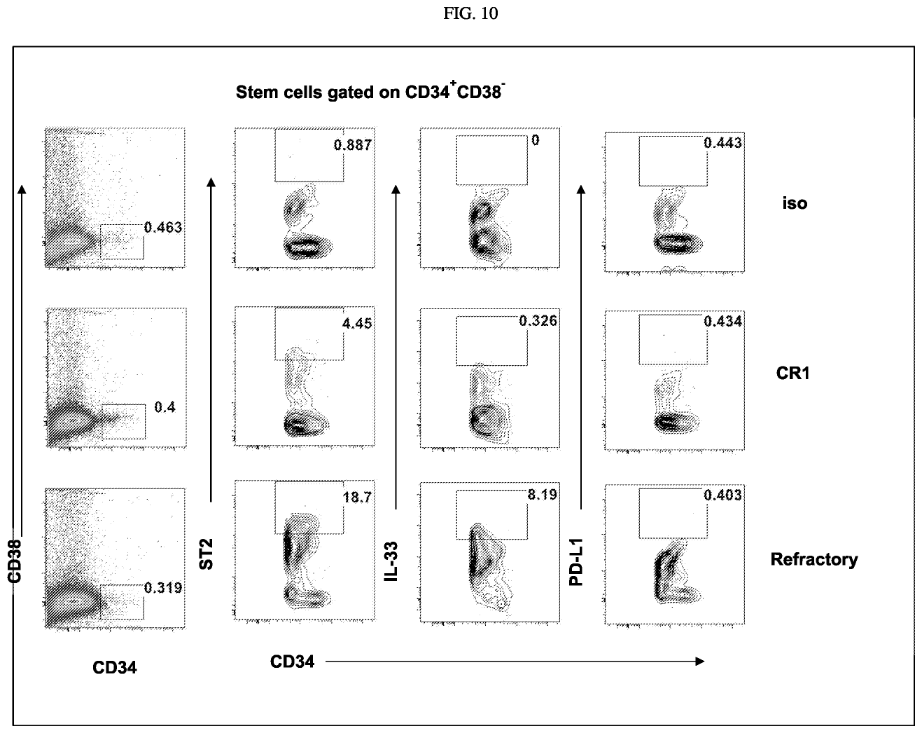

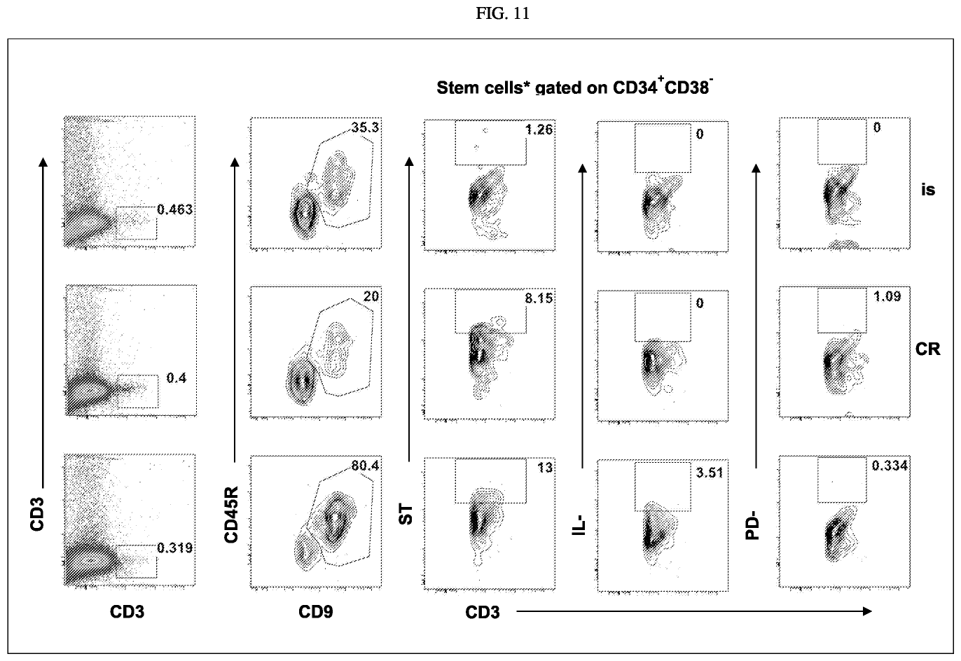

The third set of experiments comprised transcriptome/proteome analysis of intra-tumoral Tregs, immune cells, and tumoral cells and/or tissues from patients with AML after induction therapy. In these experiments, it was shown that the transcriptomic signatures of BM-derived cells Tregs in AML patients after induction chemotherapy indicated ST2 IL33 on AML cells and stem cells and more on refractory patients (see FIGS. 10 and 11).

FIG. 10 shows ST2+, IL-33+, and PDL1 expression in classical stem cells defined as CD34+ (positive) cells and CD38− (negative).The first row of FIG. 10 represents the isotype controls (“iso”). The second row of FIG. 10 is data representative of a patient with AML after chemotherapy induction and who is in complete remission, called “complete remission 1” or “CR1,” which occurs after the first round of chemotherapy. The third row is data generated from a patient with AML after chemotherapy induction and who is refractory to the chemotherapy, meaning that the patient is still leukemic after the chemotherapy. This type of patient usually does not respond to other treatment, but immunotherapy might be an option for them.

In addition, FIG. 10 shows that ST2 is already expressed in stem cells, and even moreso in the patient with refractory disease. Notably, IL-33 is not expressed in the patient with CR1, but is expressed in the patient with refractory disease. There is also no PDL-1 expression observed, which is expected on stem cells. Of note, over myeloid cells in the environment of these patients do express PDL-1.

FIG. 11 shows ST2+, IL-33+, and PDL1 expression in primitive or classical stem cells that are CD34+ (positive), CD38− (negative), CD90+, and CD45RA+ are “true” leukemic stem cells. The results of FIG. 11 show that there is less leukemic stem cells in patients in CR1 (although not totally absent), and as expected, many more leukemic cells in the refractory patient. Furthermore, ST2 is already expressed in the primitive leukemic stem cells in both the CR1 and refractory patients. However, there is more ST2 expression in the refractory patient (data not shown), while IL-33 is only expressed in the refractory patient, which has no PDl -1 expression.

In addition to the expression experiments described herein, single cell genomics and drop sequencing was also performed on actual patient samples (see the data or R050166 and RO50562 as provided herewith).

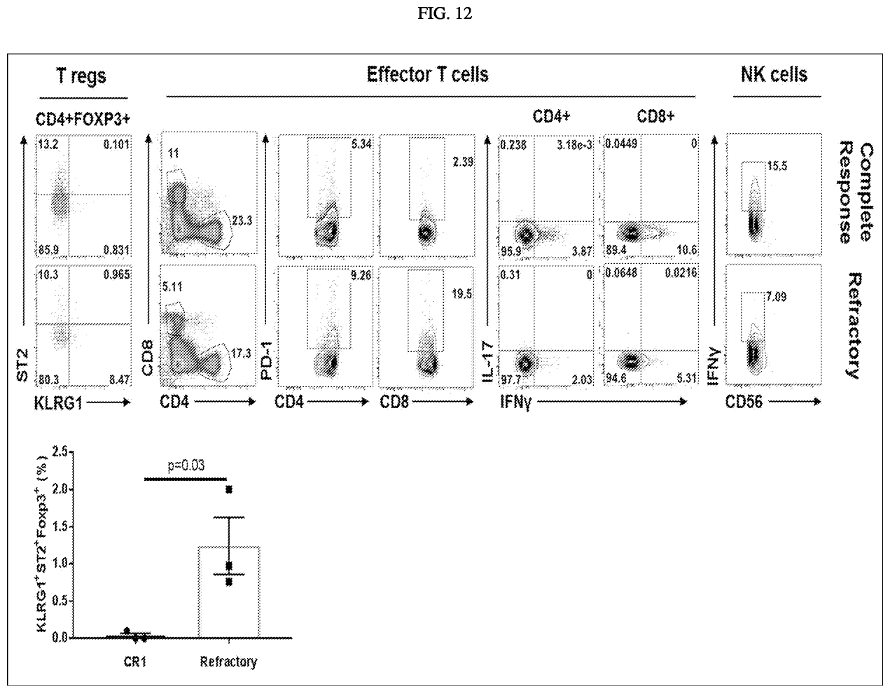

The immune signature of patients' samples from BM aspirates was focused on in human samples comparing complete response vs. refractory after the chemotherapy induction phase (FIG. 12). This inclusion of these human samples is based on the rationale that patients are seen with their first diagnosis of leukemia at different stage of the disease and often with large numbers of blasts. Deidentified biobanked samples from the FHCRC leukemia cohort (PI: Stirewalt, lab manager Era Pogosova) were used. Data are shown in FIG. 12. The frequencies of activated (KLRG1+) ST2+ Foxp3+ CD4+ Tregs is significantly increased in patients with refractory disease as compared to complete response (n=3 in each group). Although not reaching significance with only 3 samples per group, there is a trend for more PD1 on both CD4 and CD8 T cells as well as less IFN-γ producing CD4 and CD8 T cells.

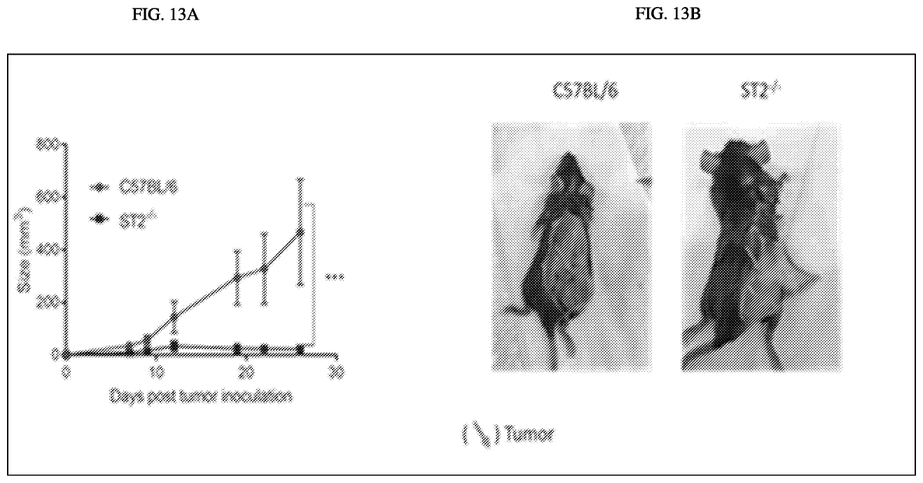

To extend the findings from hematologic tumors in solid tumors, the tumor growth of numerous murine solid tumor models such as ovarian (data not shown), pancreas (data not shown) and CRC (FIG. 13A) in WT (C57BL/6) and ST2^{-/-} mice was tested. These experiments revealed a significant tumor growth control in the CRC MC38 tumor model independently of the mouse gender (FIG. 13B). The tumor was further analyzed by flow cytometry.

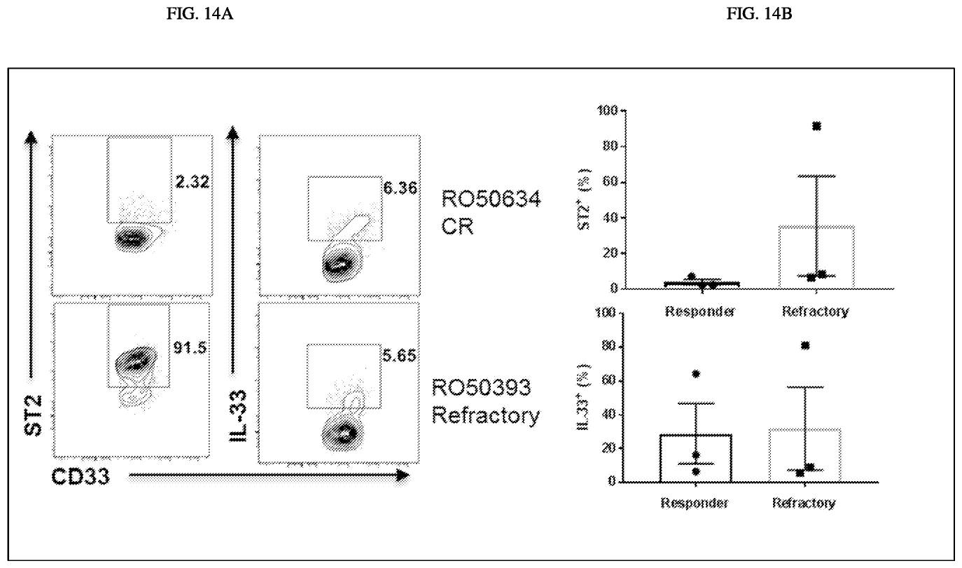

The expression of ST2 on the tumoral surface and intracellular IL-33 of various murine and human AML cells was also investigated. As shown in FIG. 14A, all cell lines tested expressed a degree of ST2 and /or IL-33. For ST2, on average 3-10% of the cells expressed ST2 but with some cell lines expressing up to 43% (Kasumi and U937). For IL-33, on average 1 to 3% but again with some cell lines expressing up to 73% (U937 and MV4-11). ST2 and IL-33 expressions are not always correlated suggesting that some tumor cells may be activated through a positive auto-feedback loop but not always. ST2 expression on AML CD45+CD33+ primary leukemic cells showing expression of ST2 on primary leukemic cells was also examined (FIG. 14B).

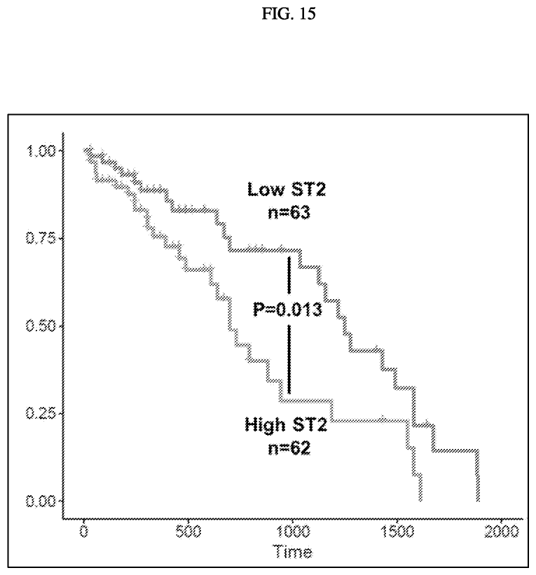

Furthermore, 125 AML cases from the cancer Genome Atlas (TCGA) dataset were associated with survival, high expression of ST2 (above the median) significantly impacted survival (p=0.013, FIG. 15). Of note, in contrast to the Kreb's group that could not show a direct expression of ST2 on the murine stem cells, it was demonstrated.

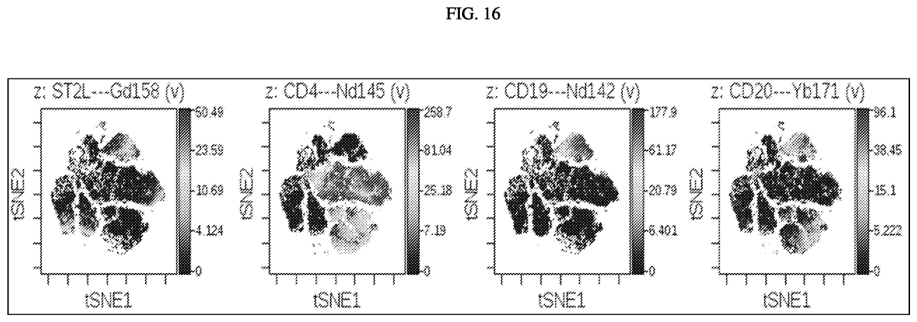

As for CRC, a next-generation deconvolution method was applied to accurately assess cell population and activities in tumor microenvironment. In contrast to what was published by the Krebs group, our analysis of TCGA databases showed that the ST2 gene is not expressed by tumoral cells. However, in 33 TCGA cancer types analyzed, 19 cancer types exhibited significant ST2 expression in at least one predicted immune/stromal cell type (˜cor>0.4). The top cell types predicted as ST2 expressing include: (1) a subset of stromal cell that express DCN, LAMA2, PODN, LUM, FBN1, COL8A2 and several other ECM components; (2) a class of immune cell expressing CD53, CD84, CD86, CYBB, DOCK2 and other immune cell markers; (3) general endothelial cell expressing CDHS, CLEC14A, TIE1 and other endothelial cell markers; and (4) T cells. These results suggest a critical role of ST2 in TME for a majority of solid tumors (FIG. 16).

These data demonstrate that ST2+ Tregs differentiate in the presence of unique cytokine combinations and that these cells are present in vivo in the BM niche and increased during AML infiltration of the BM. Further, CRC tumoral cells and AML cells express ST2. Despite these advances, there are no biomarkers currently testing these markers in the clinic. The inventions of the present disclosure are designed to address this gap.

CyTOF is a relatively novel platform for high-dimensional analysis of single cells based on specific phenotypic and functional markers. Using CyTOF, the mST2 (ST2L) antibody was optimized using healthy donors (FIG. 16). These data pointed out an ST2 positive population in other subsets than Tregs particularly B-cell subsets (FIG. 16).

These results indicate a critical role of ST2 in TME for a majority of solid tumors. More specifically, the data demonstrate that ST2+ Tregs differentiate in the presence of unique cytokine combinations and are present in vivo in the BM niche and increased during AML infiltration of the BM. Further, CRC tumoral cells and AML cells express ST2. Despite these advances, there are no biomarkers currently testing these markers in the clinic. The assays described herein are designed to address this gap.

In sum, the methods of the present disclosure indicate that specific modulation of ST2⁺ Tregs and ST2⁺ tumoral cells is a viable avenue for development as a stand-alone cancer immunotherapy, as well as one component of a combination therapy with other approved immunotherapies (e.g., anti-GD2, anti-CD33). Use of the present methods of diagnosis and treatment also work to improve response rates and mitigate the trial-and-error, as well as the toxicity associated with many cancer treatments. Therefore, the present methods are directed to methods of administering anti-ST2 antibodies to treat cancer, such as pediatric cancers, which have not shown toxicity in healthy donors and patients to date.

Moreover, the present methods demonstrate that the anti-ST2 antitumoral effect can be observed in the BM niche of mouse models, such as syngeneic HCT and tumor-bearing AML models, through inhibition of ST2+ Tregs. Therefore, the present methods are able to provide greater clarity on how ST2/IL-33 signaling potentiates the activity of Tregs and how elements of type 1 immunity counteract those effects with a specific focus on the BM niche microenvironment that has been underexplored in this context. Another important outcome of present methods is better understanding on how modulation of ST2 can influence the intratumoral Treg compartment and growth of the tumor itself.

Finally, the present methods identify the parallels between the results obtained in murine models and correlative observations in human clinical patients, such as leukemia patients whether adult or pediatric patients. The present methods ultimately provide a basis to develop personalized medicine by determining that ST2+ Tregs are significantly increased in the BM niche of patients when cancer (e.g., leukemia) develops, which aids in the decision-making of whether to deploy a therapeutic strategy blocking ST2/IL-33 signaling or enhancing the type 1 immunity through novel targets discovered and described herein, or a combination of both, as demonstrated in the dual therapeutic mechanism described herein. Combination therapies with newly discovered modulators described in the present disclosure may also improve the response rate and mitigate toxicity associated with immune checkpoint blockade. Together, the method data provided herein demonstrate the feasibility of pursuing ST2+ Tregs as targets for immunotherapeutic intervention in organisms, mouse, rat, and humans.

Notably, the present disclosure is also directed to one or more products to perform the present methods. For example, the present invention may comprise, consist essentially of, or consist of an assay, a kit, a reagent, a reactant, a chemical, a molecule, a protein, a combination thereof. In an illustrative embodiment the assay, the kit, the reagent, the reactant, the chemical, the molecule, the protein, or the combination thereof may be utilized to perform or implement the present methods, including but not limited to a method of targeting a tumor microenvironment or a method of treating cancer, as described herein.

What is claimed is:

1. A method of targeting a tumor microenvironment in a subject, the method comprising:
inhibiting ST2⁺ regulatory T cells present in the tumor microenvironment.

2. The method of claim 1, further comprising destroying ST2⁺ tumor cells located in the tumor microenvironment.

3. The method of claim 1, further comprising blocking the IL-33/ST2 pathway in the tumor microenvironment.

4. The method of claim 1, wherein the tumor microenvironment comprises tumors selected from the group consisting of liquid tumors or solid tumors.

5. The method of claim 1, wherein the tumor microenvironment comprises tumors of cancer disease.

6. The method of claim 5, wherein the cancer disease is selected from the group consisting of leukemia, lymphoma, osteosarcomas, neuroblastoma, colorectal cancer, and soft tissue sarcomas.

7. The method of claim 6, wherein the leukemia is acute myeloid leukemia (AML).

8. The method of claim 1, wherein the tumor microenvironment comprises a malignant bone marrow niche.

9. The method of claim 1, wherein the subject is an adult patient or a pediatric patient.

10. The method of claim 8, wherein the method increases tumor immunity in the malignant bone marrow niche.

11. A method of treating cancer in a subject, the method comprising:
a) administering one or more antibodies to the tumor microenvironment of the subject and
b) inhibiting ST2⁺ regulatory T cells present in the tumor microenvironment.

12. The method of claim 11, further comprising destroying ST2⁺ tumor cells located in the tumor microenvironment.

13. The method of claim 11, further comprising blocking the IL-33/ST2 pathway in the tumor microenvironment.

14. The method of claim 11, wherein the tumor microenvironment comprises tumors selected from the group consisting of liquid tumors or solid tumors.

15. The method of claim 11, wherein the tumor microenvironment comprises tumors of cancer disease.

16. The method of claim 15, wherein the cancer disease is selected from the group consisting of leukemia, lymphoma, osteosarcomas, neuroblastoma, colorectal cancer, and soft tissue sarcomas.

17. The method of claim 16, wherein the leukemia is acute myeloid leukemia (AML).

18. The method of claim 11, wherein the tumor microenvironment comprises a malignant bone marrow niche.

19. The method of claim 11, wherein the subject is an adult patient or a pediatric patient.

20. The method of claim 18, wherein the method increases tumor immunity in the malignant bone marrow niche.
